(19) United States
(12) Patent Application Publication
(10) Pub. No.: US 2002/0015951 A1
(43) Pub. Date: Feb. 7, 2002

(54) Method of analyzing a nucleic acid

(76) Inventors: Joel S. Bader, Paris (FR); Steven Gold, Branford, CT (US); Vladimir Gusev, Madison, CT (US); Shu Xia Li, Woodbridge, CT (US); Suresh Shenoy, Branford, CT (US); Oswald R. Crasta, Branford, CT (US); Pascal Boufford, Danbury, CT (US)

Correspondence Address: Ivor R. Elrifi, MINTZ, LEVIN, COHN, FERRIS,, GLOVSKY and POPEO, P.C., One Financial Center, Boston, MA, 02111, US

(21) Appl. No.: 09/755,398

(22) Filed: Jan. 4, 2001

Related U.S. Application Data
(60) Provisional application No. 60/174,685, filed on Jan. 6, 2000.

(51) Int. Cl. C12Q 1/68; G06F 19/00; G01N 33/48; G01N 33/50

(52) U.S. Cl. 435/6; 702/20

(57) ABSTRACT

Disclosed are methods of selectively analyzing a nucleic acid in a sample. The methods allow for selective identification of a target sequence in a population of nucleic acids. For example, the methods allow for confirmation of the identity of a nucleic acid tentatively identified in a quantitative expression analysis (QEA) assay.

### RELATED APPLICATIONS

This application claims priority to U.S. Ser. No. 60/174,685, filed Jan. 6, 2000. The contents of this application are incorporated by reference in their entirety.

### BACKGROUND OF THE INVENTION

As molecular biological and genetics research have advanced, it has become increasingly clear that the temporal and spatial expression of genes plays a vital role in processes occurring in both health and in disease. Moreover, the field of biology has progressed from an understanding of how single genetic defects cause the traditionally recognized hereditary disorders (e.g., the thalassemias), to a realization of the importance of the interaction of multiple genetic defects in concert with various environmental factors in the etiology of the majority of the more complex disorders, such as neoplasia.

For example, in the case of neoplasia, recent experimental evidence has demonstrated the roles of multiple defects in the expression several genes. Other complex diseases have been shown to have a similar etiology. Therefore, the more complete and reliable a correlation which can be established between gene expression and disease states, the better diseases will be able to be recognized, diagnosed and treated. This important correlation may be established by the quantitative determination and classification of DNA expression in tissue samples.

Genomic DNA (“gDNA”) sequences are those naturally occurring DNA sequences constituting the genome of a cell. The overall state of gene expression within genomic DNA (“gDNA”) at any given time is represented by the composition of cellular messenger RNA (“mRNA”), which is synthesized by the regulated transcription of gDNA. Complementary DNA (“cDNA”) sequences may be synthesized by the process of reverse transcription of mRNA by use of viral reverse transcriptase. cDNA derived from cellular mRNA also provides a representation of expressed genomic sequences within a cell at a given time. Accordingly, a method which would allow the rapid, economical and highly quantitative detection of all the DNA sequences within particular cDNA or gDNA samples is desirable.

Existing cDNA and gDNA analysis techniques are typically directed to the determination and analysis of only one or two known or unknown genetic sequences at a single time. These techniques have typically used probes which are synthesized to specifically recognize (by the process of hybridization) only one particular DNA sequence or gene. See e.g., Watson, J. 1992. Recombinant DNA, chap 7, (W. H. Freeman, New York.). Furthermore, the adaptation of these methods to the recognition of all sequences within a sample would be, at best, highly cumbersome and uneconomical.

One existing method for detecting, isolating and sequencing unknown genes uses an arrayed cDNA library. From a particular tissue or specimen, mRNA is isolated and cloned into an appropriate vector, which is introduced into bacteria (e.g., E. coli) through the process of transformation. The transformed bacteria are then plated in a manner such that the progeny of individual vectors bearing the clone of a single cDNA sequence can be separately identified. A filter “replica” of such a plate is then probed (often with a labeled DNA oligomer selected to hybridize with the CDNA representing the gene of interest) and those bacteria colonies bearing the cDNA of interest are identified and isolated. The cDNA is then extracted and the inserts contained therein is subjected to sequencing via protocols which includes, but are not limited to the dideoxynucleotide chain termination method. See Sanger, F., et al. 1977. DNA Sequencing with Chain Terminating Inhibitors. Proc. Natl. Acad. Sci. USA 74(12):5463-5467.

The oligonucleotide probes used in colony selection protocols for unknown gene(s) are synthesized to hybridize, preferably, only with the cDNA for the gene of interest. One method of achieving this specificity is to start with the protein product of the gene of interest. If a partial sequence (i.e., from a peptide fragment containing 5 to 10 amino acid residues) from an active region of the protein of interest can be determined, a corresponding 15 to 30 nucleotide (nt.) degenerate oligonucleotide can be synthesized which would code for this peptide fragment. Thus, a collection of degenerate oligonucleotides will typically be sufficient to uniquely identify the corresponding gene. Similarly, any information leading to 15-30 nt. subsequences can be used to create a single gene probe.

Another existing method, which searches for a known gene in cDNA or gDNA prepared from a tissue sample, also uses single-gene or single-sequence oligonucleotide probes which are complementary to unique subsequences of the already known gene sequences. For example, the expression of a particular oncogene in sample can be determined by probing tissue-derived cDNA with a probe which is derived from a subsequence of the oncogene's expressed sequence tag. The presence of a rare or difficult to culture pathogen (e.g., the bacterium causing tuberculosis) can also be determined by probing gDNA with a hybridization probe specific to a gene possessed by the pathogen. Similarly, the heterozygous presence of a mutant allele in a phenotypically normal individual, or its homozygous presence in a fetus, may be determined by the utilization of an allele-specific probe which is complementary only to the mutant allele. See e.g., Guo, N. C., et al. 1994. Nucleic Acid Research 22:5456-5465).

Currently, all of the existing methodologies which use single-gene probes, if applied to determine all of the genes expressed within a given tissue sample, would require many thousands to tens-of-thousands of individual probes. It has been estimated that a single human cell typically expresses approximately 5,000 to 15,000 genes, and that the most complex types of tissues (e.g., brain tissue) can express up to one-half of the total genes contained within the human genome. See Liang, et al. 1992. Differential Display of Eukaryotic Messenger RNA by Means of the Polymerase Chain Reaction. Science 257:967-971. A screening method which requires such a large number of probes can be far too cumbersome to be economic or, even practical.

In contrast, another class of existing methods, known as sequencing-by-hybridization (“SBH”), use combinatorial probes which are not gene specific. See e.g., Drmanac, et al. 1993. Science 260:1649-1652; U.S. Pat. No. 5,202,231 to Drmanac, et al. An exemplar implementation of SBH for the determination of an unknown gene requires that a single cDNA clone be probed with all DNA oligomers of a given length, say, for example, all 6 nt. oligomers. A set of oligomers of a given length which are synthesized without any type of selection is called a combinatorial probe library. A partial DNA sequence for the cDNA clone can be reconstructed by algorithmic manipulations from the hybridization results for a given combinatorial library (i.e., the hybridization results for the 4096 oligomer probes having a length of 6 nt.). However, complete nucleotide sequences are not determinable, because the repeated subsequences cannot be fully ascertained in a quantitative manner.

SBH which is adapted to the identification of known genes is called oligomer sequence signatures (“OSS”). See e.g., Lennon, et al. 1991. Trends In Genetics 7(10):314-317. OSS classifies a single clone based upon the pattern of probe “hits” (i.e., hybridizations) against an entire combinatorial library, or a significant sub-library. This method requires that the tissue sample library be arrayed into clones, wherein each clone comprises only a single sequence from the library. This technique cannot be applied to mixtures of sequences.

These previous, exemplar methodologies are all directed to finding one sequence in an array of clones—with each clone expressing a single sequence from a given tissue sample. Accordingly, they are typically not directed to rapid, economical, quantitative, and precise characterization of all the DNA sequences in a mixture of sequences, such as a particular total cellular cDNA or gDNA sample, and their adaptation to such a task would be prohibitive. Determination by sequencing the DNA of a clone, much less an entire sample of thousands of genomic sequences, may not be rapid or inexpensive enough for economical and useful diagnostics. Existing probe-based techniques of gene determination or classification, whether the genes are known or unknown, require many thousands of probes, each specific to one possible gene to be observed, or at least thousands or even tens of thousands of probes in a combinatorial library. Further, all of these aforementioned methods require the sample be arrayed into clones each expressing a single gene of the sample.

In contrast to the gene determination and classification techniques described above, another method, known as differential display, attempts to “fingerprint” a mixture of expressed genes, as is found in a pooled cDNA library. This “fingerprint,” however, seeks merely to establish whether two samples are the same or different. No attempt is made to determine the quantitative, or even qualitative, expression of particular genes. See e.g., Liang, et al. 1995. Curr. Opin. Immunol. 7:274-280; Liang, et al. 1992. Science 257:967-971; Welsh, et al. 1992. Nuc. Acid Res. 20:4965-4970; McClelland, et al. 1993. Exs. 67:103-115 and Lisitsyn, 1993. Science 259:946-950. Differential display can use the polymerase chain reaction (“PCR”) to amplify DNA subsequences of various lengths, which are then defined by their being between the annealing sites of arbitrarily selected primers. Polymerase chain reaction method and apparatus are well known. See, e.g., U.S. Pat. Nos. 4,683,202; 4,683,195; 4,965,188; 5,333,675; each herein fully incorporated by reference. Ideally, the pattern of the lengths observed is characteristic of the specific tissue from which the library was originally prepared. Typically, one of the primers used in differential display is oligo(dT) and the other is one or more arbitrary oligonucleotides which are designed to hybridize within a few hundred base pairs (bp.) of the homopolymeric poly-dA tail of a cDNA within the library. Thereby, upon electrophoretic separation, the amplified fragments of lengths up to a few hundred base pairs should generate bands which are characteristic and distinctive of the sample. In addition, changes in gene expression within the tissue may be observed as changes in one or more of the cDNA bands.

In the differential expression method, although characteristic electrophoretic banding patterns develop, no attempt is made to quantitatively “link” these patterns to the expression of particular genes. Similarly, the second arbitrary primer also cannot be traced to a particular gene due to the following reasons. First, the PCR process is less than ideally specific. One to several base pair mismatches are permitted by the lower stringency annealing step which is typically used in this method and are generally tolerated well enough so that a new chain can actually be initiated by the Taq polymerase often used in PCR reactions. Secondly, the location of a single subsequence (or its absence) is insufficient to distinguish all expressed genes. Third, the resultant bp.-length information (i.e., from the arbitrary primer to the poly-dA tail) is generally not found to be characteristic of a sequence due to: (i) variations in the processing of the 3′-untranslated regions of genes, (ii) variation in the poly-adenylation process and (iii) variability in priming to the repetitive sequence at a precise point. Therefore, even the bands which are produced often are smeared by numerous, non-specific background sequences.

Moreover, known PCR biases towards nucleic acid sequences containing high G+C content and short sequences, further limit the specificity of this method. In accord, this technique is generally limited to the “fingerprinting” of samples for a similarity or dissimilarity determination and is precluded from use in quantitative determination of the differential expression of identifiable genes.

Additional methods for establishing the repertoire of differentially expressed genes between a reference state and a state characteristic of a particular disease, pathology or experimental manipulation include the use of quantitative expression analysis (QEA). Such procedures can detect differential expression in tissue samples of portions of genomic DNA, or alternatively of mRNAs as reflected in cDNA preparations derived from such mRNAs. The QEA procedures are disclosed, for example, in U.S. Pat. No. 5,871,697, incorporated herein by reference in its entirety, as well as in Shimkets et al., “Gene expression analysis by transcript profiling coupled to a gene database query”, Nature Biotechnology 17:198-803 (1999). Briefly, QEA relies on the generation of fragments of genomic DNA or cDNA whose sequences are generally not known at the time the investigation is begun. The fragments are obtained by recognizing a certain oligomeric subsequence at one end of a double stranded nucleic acid sequence and a second oligomeric subsequence at a second end of the double stranded sequence. Such fragments are amplified and labeled using specifically designed primers and linkers that provide amplified fragments whose identifiable characteristics include a specific size provided by the length of the fragment plus an accounting of any additional bases added by the linkers, a label commonly included in the amplified fragment originating in the primer and/or the linker, and the terminal sequences at the two ends provided by the original subsequences employed at the outset as well as the nucleotides included in the linkers. The label permits detecting the presence of the fragment in a procedure intended to detect the presence of fragments, whereby the detection includes ascribing a length or size (where “length” and “size” are used interchangeably) to the fragment. After determining the size of the fragment with high precision, the complete sequence of the entire fragment is susceptible of being identified by reference to a suitable database containing a compendium of nucleic acid sequences, such that any identified sequence will include the length and terminal sequence information provided by the QEA procedure.

There are difficulties that arise in the application of QEA as just described. Such difficulties may result in ambiguity in the identification, classification or quantifying of a unique length-sequence combination for a given fragment, based on the information provided by the QEA procedure. A common difficulty is that more than one fragment fulfilling the length-subsequence combination may be provided by the sample. Alternatively, a length-subsequence combination provided by the QEA experiment may not appear in the queried database. Accordingly, in either situation, a confirmation procedure may be provided. Such a confirmation may be performed by a method whereby an amplification reaction intended to amplify a given QEA fragment, with its known subsequence and linker information, is carried out in two separate samples, one in the absence of any competing primers, and a second in the presence of primers and/or linkers which provide the same subsequence and linker information as the first, but include no labeled components. The presence of the unlabeled components competes effectively for the labeled components, and provides amplification products that are not detectable in the QEA procedure. This procedure may be called “poisoning” herein, and is intended to confirm a fragment identified in a QEA experiment by reducing part or all of the ambiguity referred to above. Poisoning has been disclosed in WO99/07896, and is incorporated herein by reference in its entirety.

### SUMMARY OF THE INVENTION

The invention is based in part on the discovery of a highly sensitive and accurate method for identifying a candidate sequence in a population of nucleic acids using an improvement of an oligo-competition QEA procedure. The invention allows for the enhancement of the QEA selection process.

In one aspect, the invention features a method for identifying, classifying or quantifying one or more nucleic acids in a sample comprising a plurality of nucleic acids having different nucleotide sequences. The method includes probing the sample with one or more recognition means wherein each recognition means recognizes a different target nucleotide subsequence or a different set of target nucleotide subsequences to provide one or more targeted nucleic acids. One or more first signals is generated from the sample probed by the recognition means. Each generated first signal arises from a targeted nucleic acid in the sample and includes a representation of (i) the length between occurrences of target subsequences in the targeted nucleic acid, and (ii) the identities of the target subsequences in the targeted nucleic acid or identities of the target subsequences among which are included the target subsequences in the targeted nucleic acid. One or more targeted nucleic acids are selected based on their corresponding first signals.

Sequence information from one or more target subsequences in the selected targeted nucleic acid is extended by one or more nucleotides providing one or more extended subsequences under conditions that generate one or more second signals arising from the selected targeted nucleic acid, wherein at least one of the selected targeted nucleic acids has been extended, in the sample. The second signal comprises a representation of (i) the length between occurrences of target subsequences, at least one of which has been extended, in the nucleic acid, and (ii) the identities of the selected target subsequences, at least one of which has been extended, in the selected targeted nucleic acid or identities of the target subsequences, at least one of which has been extended, among which are included the target subsequences in the selected targeted nucleic acid.

A nucleotide sequence database is then searched to determine sequences that match or the absence of any sequences that match one or more or of the selected targeted nucleic acids having at least one extended subsequence and represented by the generated second signals. The database includes a plurality of known nucleotide sequences of nucleic acids that may be present in the sample. A sequence from the database is determined to match the selected targeted nucleic acid providing a generated second signal when the sequence from the database has both (i) the same length between occurrences of target subsequences, at least one of which has been extended, as is represented by the generated signal, and (ii) the same target subsequences, at least one of which has been extended, as are represented by the generated signal, or target subsequences, at least one of which has been extended, that are members of the same sets of target subsequences represented by the generated signal.

The second generated signal can be, e.g., a negative oligo-competition signal or a positive oligo-competition signal.

In some embodiments, extension of the sequence information includes contacting the nucleic acid sample with a mixture of oligonucleotides comprising (i) a set of labeled primers each of whose nucleotide sequences comprises a target subsequence and (ii) an unlabeled primer whose sequence comprises one of the target subsequences identified in (i) followed by at least one additional nucleotide. In desired extension of the sequence information can include contacting the nucleic acid sample with a mixture of oligonucleotides comprising (i) a set comprising a first unlabeled primer and a second unlabeled primer each of whose nucleotide sequence comprises a target subsequence and (ii) a set comprising a labeled third primer whose sequence comprises the subsequence of the first unlabeled primer and a labeled fourth primer whose sequence comprises the subsequence of the second unlabeled primer extended by at least one nucleotide.

In some embodiments, at least one of the generated signals corresponds to a sequence having a size and target subsequence of a sequence present in the sequence database.

In some embodiments, the method additionally includes recovering a fragment of a nucleic acid in the sample which generates the signal, sequencing the fragment to determine at least a partial sequence for the fragment, and verifying that the sample comprises a nucleic acid having a sequence comprising at least a portion of the determined sequence.

In preferred embodiments, the plurality of nucleic acids is DNA. The method can include digesting the sample with one or more restriction endonucleases, wherein the restriction endonucleases have recognition sites that are the target subsequences and leave single-stranded nucleotide overhangs on the digested ends, hybridizing double-stranded adapter nucleic acids with the digested sample fragments, the adapter nucleic acids having an end complementary to one of the single-stranded overhangs; and ligating the complementary ends of adapter nucleic acids to the complementary 5′-end of a strand of the digested sample fragments to form ligated nucleic acid fragments.

In some embodiments the extended subsequences are unlabeled.

In a further aspect, the invention is a method for extending the sequence in a length-subsequence combination of one or more nucleic acids in a sample comprising a plurality of nucleic acids having different nucleotide sequences. The method includes probing the sample with one or more recognition means. Each recognition means recognizes a different target nucleotide subsequence or a different set of target nucleotide subsequences to provide one or more targeted nucleic acids.

One or more first signals from the sample probed by the recognition means is generated. Each generated first signal arises from a targeted nucleic acid in the sample and includes a representation of (i) the length between occurrences of target subsequences in the targeted nucleic acid, and (ii) the identities of the target subsequences in the targeted nucleic acid or identities of the target subsequences among which are included the target subsequences in the targeted nucleic acid. One or more targeted nucleic acids based on their corresponding first signals is selected, and sequence information from one or more target subsequences in the targeted nucleic acid is extended by one or more nucleotides providing one or more extended subsequences.

Extension is performed under conditions that generate one or more second signals arising from selected targeted nucleic acids in the sample at least one of whose subsequences has been extended. The second signal includes a representation of (i) the length between occurrences of target subsequences, at least one of which has been extended, in the nucleic acid, and (ii) the identities of the target subsequences, at least one of which has been extended, in the selected targeted nucleic acid or identities of the target subsequences, at least one of which has been extended, among which are included the target subsequences in the selected targeted nucleic acid. A matched nucleic acid in the sample has an extended sequence in the length-subsequence combination.

In some embodiments, the second generated signal is a negative oligo-competition signal. In other embodiments, the second generated signal is a positive oligo-competition signal.

In some embodiments, extension of the sequence information comprises contacting the nucleic acid sample with a mixture of oligonucleotides comprising (i) a set of labeled primers each of whose nucleotide sequences comprises a target subsequence and (ii) an unlabeled primer whose sequence comprises one of the target subsequences identified in (i) followed by at least one additional nucleotide. For example, extension of the sequence information can include contacting the nucleic acid sample with a mixture of oligonucleotides comprising (i) a set comprising a first unlabeled primer and a second unlabeled primer each of whose nucleotide sequence comprises a target subsequence and (ii) a set comprising a labeled third primer whose sequence comprises the subsequence of the first unlabeled primer and a labeled fourth primer whose sequence comprises the subsequence of the second unlabeled primer extended by at least one nucleotide.

Among the advantages of the present invention are that it allow for highly accurate and sensitive identification, classification or quantifying of subsequences in a sample of genomic DNA or cDNA (such that the subsequences present in the resulting fragment differ from the intended subsequences). The invention also allows for precise linking and/or priming in the QEA procedure. In addition, the method provides for sizing procedures that resolve fragments of similar but different sizes, and related experimental difficulties.

The invention in addition allows for unambiguous identification of terminal subsequences in QEA analyses. In addition, it allows for performing QEA procedures in which a single gene fragment is provided without sequence ambiguities.

All technical and scientific terms used herein have the same meanings commonly understood by one of ordinary skill in the art to which this invention belongs. Although any methods and materials similar or equivalent to those described herein can be used in the practice of the present invention, the preferred methods and materials are now described. The citation or identification of any reference within this application shall not be construed as an admission that such reference is available as prior art to the present invention. All publications mentioned herein are incorporated herein in their entirety by reference.

### BRIEF DESCRIPTION OF THE DRAWINGS

FIG. 1 is a diagram describing the different parts of a QEA fragment.

FIG. 2 is a graph showing a trace of fluorescent intensities plotted against size of the fluorescent fragment in base pairs.

FIG. 3 is a diagram describing a principle of an oligonucleotide competition assay.

FIG. 4 is a graph showing a trace of fluorescent intensities blotted against size in base pairs for control and oligo-competed samples.

FIG. 5 is a diagram illustrating a oligonucleotide competition reaction.

FIG. 6 is a diagram containing two traces. The top traces plot the fluorescence of four different oligo competition reactions using four different versions of the R primer. These four versions vary in the nucleotide just 3′ of the restriction site. The bottom traces plot similar data for the J primer.

FIG. 7 is a diagram containing two traces. The top traces plot the fluorescence of four different oligo competition reactions using four different versions of the R primer. These four versions vary in the second nucleotide 3 ′ of the restriction site. The bottom traces plot similar data for the J primer.

FIG. 8 is a graph showing two traces of fluorescence plotted against nucleic acid size of a control and oligo-competed samples.

FIG. 9 is a graph showing four traces of fluorescence plotted against nucleic acid size.

FIG. 10 is a graph showing four traces of fluorescence against nucleic acid size.

FIG. 11 is a graph showing the overall association of the trace poisoning score and trace poisoning effectiveness compared to historical data.

FIG. 12 is a graph showing the overall association of the trace poisoning score and poisoning success among GeneCalls™ from a sequence database.

### DETAILED DESCRIPTION OF THE INVENTION

The present invention provides a method for extending the known subsequences at one or both of the two ends of double stranded QEA fragment inward in the 3′ direction by an additional number of nucleotide positions. The general method disclosed herein may be designated “oligo-competition,” “extended oligo-competition,” or “trace oligo-competition”, or similar terms. The extension is accomplished by using as the primer in the amplification step a competing oligonucleotide including an additional base at the 3′ end of the originally known subsequence. Such an oligonucleotide is termed an “extended” oligonucleotide herein. Since the identity of the base 3′ to the known subsequence is initially unknown, it may be any one of the four possible naturally occurring bases, A, C, G, or T. Four separate oligo-competition runs are carried out in parallel, each having either A, C, G, or T at the 3′ end of the priming oligonucleotide. Since these are unlabeled, the particular one of the four extended oligonucleotide primers providing the diminution or obliteration of the detection of the fragment targeted by the extended oligonucleotides identifies the correct additional base at the 3′ end of the original subsequence.

The known subsequences may have any length, based on ways known in the art for identifying the subsequences in a sample of genomic DNA or cDNA. In preferred embodiments these known subsequences are provided by the recognition sequences of various restriction endonucleases. Thus, for example, subsequence lengths may range from about 4 nucleotides up to as many as 8 nucleotides. The operation of one cycle of the extended oligo-competition of the present invention at one end of a fragment thus extends the length of the known subsequence by one nucleotide; for example, an initial known subsequence of four bases becomes an extended known sequence of 5 bases, or an initial known subsequence of 8 bases becomes an extended known sequence of 9 bases. This procedure reduces the ambiguity in the final extended subsequence by a factor of 4 for each cycle at each end of the fragment.

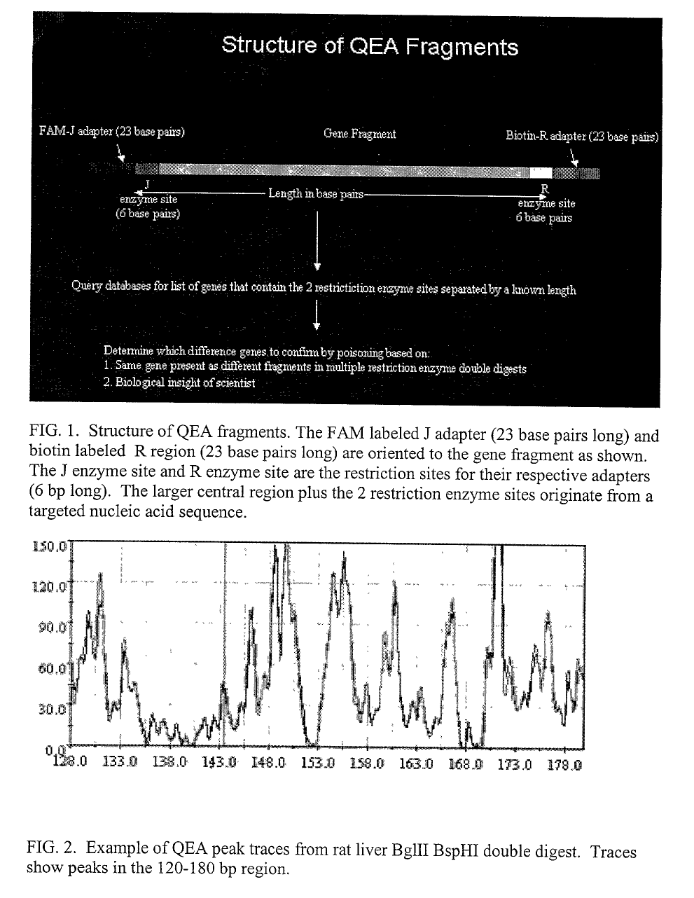

FIG. 1 depicts a double stranded QEA fragment prepared by the PCR procedure described herein. The fragment is labeled at one end, on one strand by a FAM label to facilitate detection, and on the second end, on the complementary strand, by a biotin label to facilitate isolation. The subsequences specific for each end are called the J subsequence, corresponding to the recognition sequence for a J-specific restriction endonuclease, and the R subsequence, corresponding to the recognition sequence for an R-specific restriction endonucleases.

An important embodiment of extended oligo-competition may be described as follows (see FIG. 1). The QEA process (alternatively termed “GeneCalling™” herein) involves a) fragmentation of cDNA pools with two different restriction enzymes, b) ligation of the restriction fragments to a FAM-labelled DNA adapter (the J adapter) at one end of the fragment and a biotin-labeled DNA adapter (the R adapter) at the second end of the fragment; c) polymerase chain reaction (PCR) amplification of the ligated DNA molecules using primers specific to the sequences contained within the 2 adapter modules, which leads to the production of approximately 300 fluorescent DNA fragments (called quantitative expression analysis bands, or QEA bands); d) purification of the biotin-labeled fragments on streptavidin-coated magnetic beads; and e) determination of the size of the fragments by capillary electrophoresis of the purified QEA bands in the presence of a sizing ladder. The electrophoresis step provides the length (in base pairs within 0.2 bp) of the sequence included in each fragment in the original cDNA pool as well as its precise abundance (as the peak height). Based on the length of the cDNA restriction fragment and the identity of the two restriction enzymes used to generate it, a list of potential genes is developed by querying known and proprietary databases for genes predicted to possess this restriction fragment.

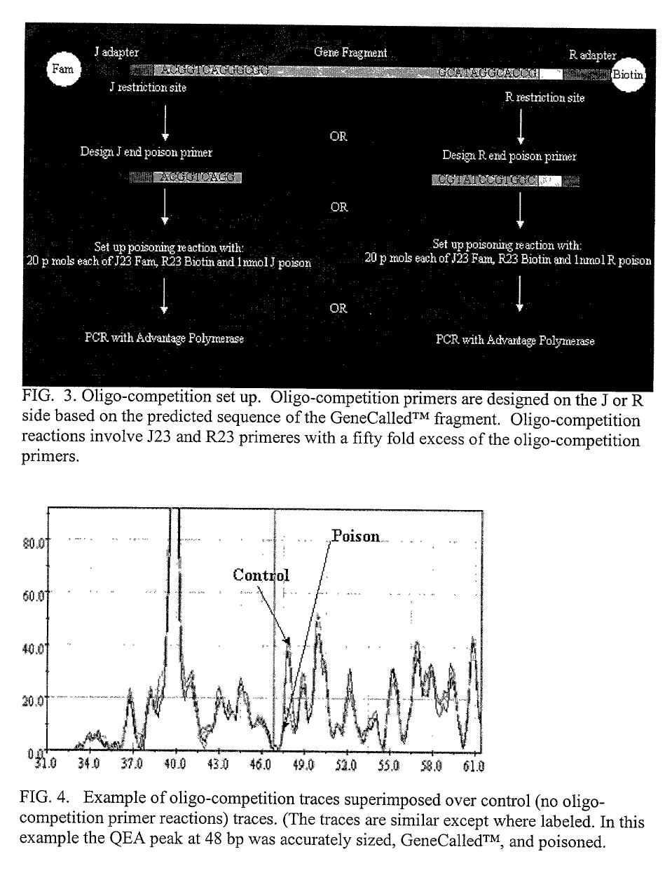

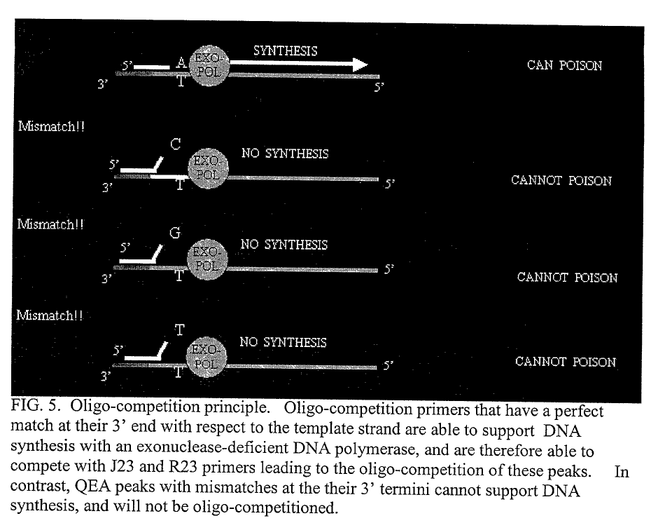

Confirmation of a band's identity involves a competitive PCR reaction using the QEA bands described above with three primers (see FIG. 3): a FAM-labeled primer, J23, a biotin-labeled primer, R23, and a 50 fold molar excess of a third, unlabelled primer known as a oligo-competition primer. The oligo-competition primer shares a 5-base overlap with either the J (in the case of the J oligo-competition primer) or R primer (in the case of the R oligo-competition primer) at its 5′ end, followed by the restriction enzyme subsequence, and a 9-11 nucleotides region that contains gene-specific sequences at its 3′ end. The latter sequences originate from the gene identification provided after the database lookup step described in the preceding paragraph. The competition between Fam-labeled J23 and J-oligo-competition primers to participate in the PCR reaction with the R23 primer involves only the GeneCalled™ peak in the milieu of approximately 300 other QEA fragments. Thus, if the GeneCall is accurate, then the design of the oligo-competition primer would provide an unlabeled PCR product for a specific peak, while the production of all other Fam labeled peaks is unaffected. Oligo-competition PCR reactions are visualized by comparing the oligo-competition PCR fluorescent traces to their non-competed counterparts (products of a PCR reaction using only the Fam-J23 and biotin-R23 primers) counterparts. In a successful oligo-competition, all peaks are recapitulated in both traces except for the peak for which the oligo-competition primer was designed. When the GeneCall of a QEA peak are inaccurate, the primer designed is specific to a gene different from that which is contained in the peak. Therefore, in an unsuccessful oligo-competition reaction, both the oligo-competed and non-oligo-competed traces are identical.

The present invention describes new oligo-competing primers (called oligo-competition primers) that extend, in a given cycle of the method, only one nucleotide into the cDNA in order to determine the identity of that nucleotide (see FIG. 5). Therefore, in a oligo-competition reaction (called a phasing reaction) in which the base at the 3′ end of the chosen subsequence is, for example, an A, phasing reactions are conducted using oligo-competition primers have either in A, G, C, or T nucleotides at their 3′ ends to determine which QEA peaks correspond to fragments having an A at their 3′ prime ends, all peaks that have this nucleotide as its first nucleotide 3′ of the restriction site will be poisoned by the unlabeled primer, and so remain undetected. Only the reaction competed by the oligo-competing primer with A at its 3′ end will provide a diminished signal, and so A will be identified as the next 3′ nucleotide. By setting up 4 parallel phasing reactions (one each with oligo-competition primers that end in A, C, G, or T at the 3′ end) on the J restriction side, and an additional 4 parallel phasing reactions on the R end, the identity of the nucleotide on 3′ side of both restriction enzyme recognition subsequences can be determined. In further cycles, the nucleotides at the second positions removed from the 3′ end of each restriction enzyme subsequence site may also be identified by conducting phasing reactions similar to ones described above by using oligo-competition primers that have the dinucleotide XA, XC, XG, or XT at their 3′ ends, where X here represents the particular base already identified in the preceding cycle. Alternatively, the first nucleotide position may be occupied by any of the four bases, namely, NA, NC, NG, or NT at their 3′ ends, where N here represents any nucleotide, or a mixture of the four nucleotides. N may also be an ambiguous base or a universally-pairing base such as I. In the present discussion, operation of the method for two cycles at each of the J and R sites of the fragment targeted by the oligo-competing primers provides the identity of four additional nucleotides (the 2 nucleotides 3′ of the J restriction enzymes site, and the 2 nucleotides 3′ of the R restriction site). Accordingly, the ambiguity in identifying a fragment as originating from a given gene GeneCall™ list for each peak is refined by a factor of 4⁴, or 256, leading to a nearly unique subsequence-length combination, permitting essentially unambiguous gene identification of the restriction fragment.

### EXAMPLES

The invention will be further illustrated in the following examples, which do not limit the scope of the attached claims.

### Example 1

### Restriction Endonuclease Properties Used in the Extended Oligo-Competition Method

Table 1 shows all the restriction enzymes tested and their modules that were used in primer design. The modules presented in Table 1 are the single strand overhangs resulting from the asymmetric cleavage catalyzed by the given endonucleases.

TABLE 1
List of restriction modules tested

| Restriction Enzyme | Restriction enzyme site module |
|---|---|
| Acc65I | GTACC |
| ApaLI | TGCAC |
| ApoI | AATTY |
| AvrII | CTAGG |
| BamHI | GATCC |
| BclI | GATCA |
| BglII | GATCT |
| BspEI | CCGGA |
| BspHI | CATGA |
| BsrFI | CCGGY |
| BsrGI | GTACA |
| BstYI | GATCY |
| EaeI | GGCCR |
| EagI | GGCCG |
| EcoRI | AATTC |
| HindIII | AGCTT |
| MfeI | AATTG |
| MluI | CGCGT |
| NcoI | CATGG |
| NgoMIV | CCGGC |
| NheI | CTAGC |
| PspOMI | GGCCC |
| SpeI | CTAGT |
| XbaI | CTAGA |
| XhoI | TCGAG |

Table 2 shows all the restriction enzyme pairs tested along with the identification of which restriction enzyme sites are on the J or the R side.

TABLE 2
List of restriction enzyme double digests tested

| Restriction enzymes | J-side restriction | R-side restriction |
|---|---|---|
| in double digest | enzyme | enzyme |
| Acc65I/ BglII | Acc65I | BglII |
| Acc65I/ BclI | Acc65I | BclI |
| Ac65I/ MfeI | Acc65I | MfeI |
| Acc65I/ BspEI | Acc65I | BspEI |
| EagI/ BglII | EagI | BglII |
| BclI/ BspEI | BclI | BspEI |
| BclI/ ApaLI | BclI | ApaLI |
| BglII/ BspEI | BglII | BspEI |
| BglII/ ApaLI | BglII | ApaLI |
| BglII/ MfeI | BglII | MfeI |
| BglII/ MluI | BglII | MluI |
| BsrGI/ MfeI | BsrGI | MfeI |
| BsrGI/ BglII | BsrGI | BglII |
| BsrGI/ BspEI | BsrGI | BspEI |
| BsrGI/ BclI | BsrGI | BclI |
| BamHI/ BspHI | BamHI | BspHI |
| BamHI/ HindIII | BamHI | HindIII |
| BamHI/ BsrGI | BamHI | BsrGI |
| BamHI/ ApaLI | BamHI | ApaLI |
| EcoRI/ BclI | EcoRI | BclI |
| EcoRI/ PspOMI | EcoRI | PspOMI |
| EcoRI/ BglII | EcoRI | BglII |
| EcoRI/ Acc65I | EcoRI | Acc65I |
| EcoRI/ NgoMIV | EcoRI | NgoMIV |
| EcoRI/ ApaLI | EcoRI | ApaLI |
| EcoRI/ XhoI | EcoRI | XhoI |
| EcoRI/ NheI | EcoRI | NheI |
| HindIII/ MfeI | HindIII | MfeI |
| HindIII/ BglII | HindIII | BglII |
| HindIII/ BclI | HindIII | BclI |
| HindIII/ EcoRI | HindIII | EcoRI |
| HindIII/ NheI | HindIII | NheI |
| HindIII/ BsrGI | HindIII | BsrGI |
| HindIII/ XbaI | HindIII | XbaI |
| HindIII/ SpeI | HindIII | SpeI |
| HindIII/ AvrII | HindIII | AvrII |
| HindIII/ BspHI | HindIII | BspHI |
| HindIII/ MluI | HindIII | MluI |
| HindIII/ ApaLI | HindIII | ApaLI |
| HindIII/ NcoI | HindIII | NcoI |
| HindIII/ NgoMIV | HindIII | NgoMIV |
| BspEI/ BspHI | BspEI | BspHI |
| BspEI/ EcoRI | BspEI | EcoRI |
| BspEI/ NcoI | BspEI | NcoI |
| BspEI/ MfeI | BspEI | MfeI |
| BspEI/ NheI | BspEI | NheI |
| BspEI/ ApaLI | BspEI | ApaLI |
| BspEI/ BamHI | BspEI | BamHI |
| BspEI/ BstYI | BspEI | BstYI |
| BspEI/ EaeI | BspEI | EaeI |
| BspEI/ PspOMI | BspEI | PspOMI |
| BspEI/ SpeI | BspEI | SpeI |
| NheI/ BclI | NheI | BclI |
| NheI/ BglII | NheI | BglII |
| NheI/ NcoI | NheI | NcoI |
| NheI/ ApaLI | NheI | ApaLI |
| NheI/ Acc65I | NheI | Acc65I |
| NheI/ BsrGI | NheI | BsrGI |
| AvrII/ BamHI | AvrII | BamHI |
| AvrII/ BclI | AvrII | BclI |
| AvrII/ NcoI | AvrII | NcoI |
| AvrII/ PspOMI | AvrII | PspOMI |
| AvrII/ BspEI | AvrII | BspEI |
| AvrII/ Acc65I | AvrII | Acc65I |
| AvrII/ BglII | AvrII | BglII |
| AvrII/ ApoI | AvrII | ApoI |
| AvrII/ BsrGI | AvrII | BsrGI |
| BsrF1/ BglII | BsrF1 | BglII |
| ApoI/ NgoMIV | ApoI | NgoMIV |
| ApoI/ BamHI | ApoI | BamHI |
| ApoI/ BspEI | ApoI | BspEI |
| ApoI/ BstYI | ApoI | BstYI |
| XbaI/ BsrGI | XbaI | BsrGI |
| XbaI/ NcoI | XbaI | NcoI |
| XbaI/ BglII | XbaI | BglII |
| XbaI/ BstYI | XbaI | BstYI |
| XbaI/ PspOMI | XbaI | PspOMI |
| XbaI/ BamHI | XbaI | BamHI |
| BspHI/ EcoRI | BspHI | EcoRI |
| BspHI/ BglII | BspHI | BglII |
| BspHI/ NgoMIV | BspHI | NgoMIV |
| BspHI/ Acc65I | BspHI | Acc65I |
| SpeI/ ApoI | SpeI | ApoI |
| SpeI/ BclI | SpeI | BclI |
| SpeI/ BglII | SpeI | BglII |
| SpeI/ BsrGI | SpeI | BsrGI |
| SpeI/ MfeI | SpeI | MfeI |
| SpeI/ BstYI | SpeI | BstYI |
| SpeI/ BamHI | SpeI | BamHI |
| SpeI/ BspHI | SpeI | BspHI |
| SpeI/ PspOMI | SpeI | PspOMI |
| NcoI/ BglII | NcoI | BglII |
| NcoI/ Acc65I | NcoI | Acc65I |
| MfeI/ NheI | MfeI | NheI |
| MfeI/ BamHI | MfeI | BamHI |
| EaeI/ MfeI | EaeI | MfeI |
| ApaLI/ SpeI | ApaLI | SpeI |
| BstYI/ Acc65I | BstYI | Acc65I |
| BstYI/ ApaLI | BstYI | ApaLI |
| MluI/ BstYI | MluI | BstYI |
| MluI/ EcoRI | MluI | EcoRI |
| MluI/ ApoI | MluI | ApoI |
| PspOMI/ BsrGI | PspOMI | BsrGI |

### Example 2

### Extended Oligo-Competition Applied to a QEA Digest

Primer Design (see FIG. 1)

1. Fam-labeled J23: the J23 primer is labeled with Fam at its 5′ end and has the following sequence: 5′ ACCGACGTCGACTATCCATGAAG 3′ (SEQ ID NO:1).

2. The biotin-R23 primer is labeled with biotin at its 5′ end and has the following sequence: 5′ AGCACTCTCCAGCCTCTCACCGA 3′ (SEQ ID NO:2).

Oligo-competition primers. Competing primers are unlabeled oligonucleotides composed of a 3′ portion of the J-adapter or R-adapter (FIG. 1), fused to a module given by the last 5 nucleotides of the restriction enzyme subsequence, and ending in the 1 or 2 discriminating bases (see Table 3). Specifically, the sequences of the J-end oligo-competition primers, starting at the 5′ end, share the last 14 nucleotides at the 3′ end of J joined to the last 5 nucleotides of the restriction enzyme recognition sequence. They end in one of the four discriminating nucleotides (A, C, G, or T) for use in a first cycle of competing. Phasing primers that investigate the identity of the nucleotide 2 bases removed from the 3′ end of the restriction enzyme recognition sequence have an ambiguous mixture of nucleotides (an equimolar mix of the 4 nucleotides, or N) at the 3′ penultimate position of the oligo-competition primer followed by one of the four discriminating nucleotides (A, C, G, or T) at the 3′ end. The 16 oligo-competition primers required for extracting 4 base information from a QEA reaction involving the restriction enzymes BspHI and BglII is shown in Table 3; in this example the first cycle applies competing primers that are 21 bases in length, and the second cycle applies competing primers that are 22 bases long.

TABLE 3
Primers required for phasing at 4 positions for QEA peaks from a
BspHI (TCATGA) and BglII (AGATCT) double restriction enzyme digest

| | | Last 5 | | |
|---|---|---|---|---|
| | | nucleo- | | |
| | | tides | Dis- | |
| | | of | crim- | |
| | | restric- | inat- | |
| | | tion | ing | |
| | | enzyme | nuc- | Position in QEA |
| Nomen- | J or R sequence | site | leo- | fragment being |
| clature | module | Module | tides | phased |
| M0J1A | GACTATCCATGAAGA | CATGA | A | 1^{st} base 3' of J |
| | (SEQ ID NO:3) | | | side restriction |
| | | | | site |
| M0J1C | GACTATCCATGAAGA | CATGA | C | 1^{st} base 3′ of J |
| | (SEQ ID NO:3) | | | side restriction |
| | | | | site |
| M0J1G | GACTATCCATGAAGA | CATGA | G | 1^{st} base 3′ of J |
| | (SEQ ID NO:3) | | | side restriction |
| | | | | site |
| M0J1T | GACTATCCATGAAGA | CATGA | T | 1^{st} base 3′ of J |
| | (SEQ ID NO:3) | | | side restriction |
| | | | | site |
| M0J2A | GACTATCCATGAAGA | CATGA | NA | 2nd base 3′ of J |
| | (SEQ ID NO:3) | | | side restriction |
| | | | | site |
| M0J2C | GACTATCCATGAAGA | CATGA | NC | 2nd base 3′ of J |
| | (SEQ ID NO:3) | | | side restriction |
| | | | | site |
| M0J2G | GACTATCCATGAAGA | CATGA | NG | 2nd base 3′ of J |
| | (SEQ ID NO:3) | | | side restriction |
| | | | | site |
| M0J2T | GACTATCCATGAAGA | CATGA | NT | 2nd base 3′ of J |
| | (SEQ ID NO:3) | | | side restriction |
| | | | | site |
| I0R1A | CAGCCTCTCACCGAC | GATCT | A | 1^{st} base 3′ of R |
| | (SEQ ID NO:4) | | | side restriction |
| | | | | site |
| I0R1C | CAGCCTCTCACCGAC | GATCT | C | 1^{st} base 3′ of R |
| | (SEQ ID NO:4) | | | side restriction |
| | | | | site |
| I0R1G | CAGCCTCTCACCGAC | GATCT | G | 1^{st} base 3′ of R |
| | (SEQ ID NO:4) | | | side restriction |
| | | | | site |
| I0R1T | CAGCCTCTCACCGAC | GATCT | T | 1^{st} base 3′ of R |
| | (SEQ ID NO:4) | | | side restriction |
| | | | | site |
| I0R2A | CAGCCTCTCACCGAC | GATCT | NA | 2nd base 3′ of R |
| | (SEQ ID NO:4) | | | side restriction |
| | | | | site |
| I0R2C | CAGCCTCTCACCGAC | GATCT | NC | 2nd base 3′ of R |
| | (SEQ ID NO:4) | | | side restriction |
| | | | | site |
| I0R2G | CAGCCTCTCACCGAC | GATCT | NG | 2nd base 3′ of R |
| | (SEQ ID NO:4) | | | side restriction |
| | | | | site |
| I0R2T | CAGCCTCTCACCGAC | GATCT | NT | 2nd base 3′ of R |
| | (SEQ ID NO:4) | | | side restriction |
| | | | | site |

Phasing Reactions

Phasing reactions were conducted with 1 ng of QEA reaction products, 100 pmol each of Fam-J23 and biotin R-23 primers, 1 nmol of the appropriate J or R oligo-competition primers in a buffer that contains 10 mM KCl, 10 mM NaCl, 22 mM Tris-HCl, pH 8.8, 10 mM (NH₄)₂SO4, 2 mM MgSO4, 2 mM MgCl₂, 0.2 mM dithiothreitol, 100 mM betaine (Sigma) 0.1% Triton X-100, 0.4 mM of each dNTP, and 0.8 units of Deep Vent (exo-) DNA polymerase (New England Biolabs). The PCR program used for the reactions was 96° C. for 5 min, followed by 13 cycles of 95° C. for 30 s, 57° C. for 1 min, and 72° C. for 2 mins. The reactions were finished by a step at 72° C. for 10 mins.

Oligo-competition products were purified using magnetic streptavidin coated beads, denatured by heating to 95° C. for 5 min to release the strand labeled with Fam, mixed with a Rox labeled DNA sizing ladder and subjected to capillary electrophoresis for size determination using the MegaBace 1000 system (Molecular Dynamics).

Results

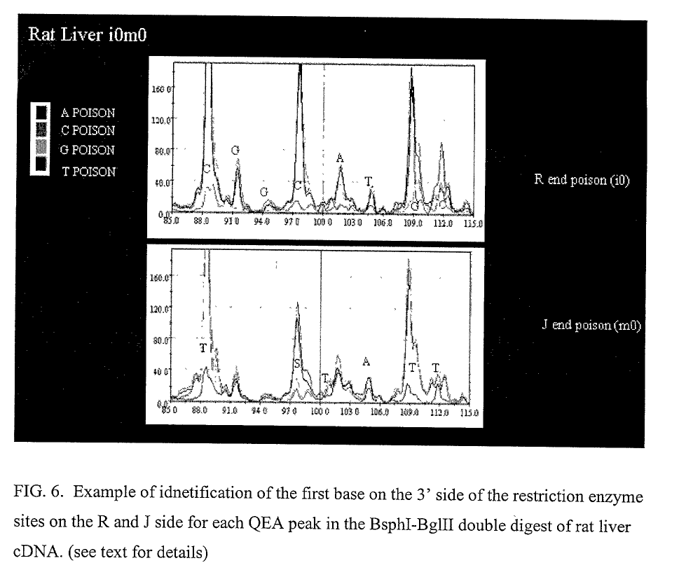

Oligo-competition reactions were conducted using rat liver QEA reactions from a BspHI-BglII double digest. For the first extended position on the J side 4 reactions were conducted, each employing 100 pmols of J23 and R23 primers and, using the nomenclature provided in Table 3, 1 nmol of either M0J1A, M0J1C, M0J1G, or M0J1T. Similarly for the first position on the R side we conducted four additional reactions that involved 100 pmols each of J23 and R23 primers and 1 nmol of either I0R1A, I0R1C, I0R1G, or I0R1T. The PCR reactions were conducted, purified and subjected to capillary electrophoresis. The traces from each reaction on the J side and R side are shown in FIG. 6.

The four traces in the top panel of FIG. 6 correspond to QEA peaks obtained after competition reactions involving the I0R1A, I0R1C, I0R1G, and I0R1T oligo-competition primers respectively. Similarly, the bottom panel shows the QEA peaks that are obtained after oligo-competition reactions with the M0J1A, M0J1C, M0J1G, and M0J1T primers. The trace with the lowest height for a given peak identifies the nucleotide on the 3′ side of the restriction enzyme site. For example, in this region of the trace, the peak at 88.2 bp has a cytosine (C) residue 3′ of the BglII site (FIG. 6, top panel), and a thymine (T) residue on the 3′ side of the BspHI site (FIG. 6, bottom panel). Similar designations in the panels of FIG. 6 indicate the base providing the successful competition for other QEA peaks in the sized detection. (The designation “S” in the bottom panel of FIG. 6 designates the ambiguity that C and G both appear to compete successfully at this position of this fragment.)

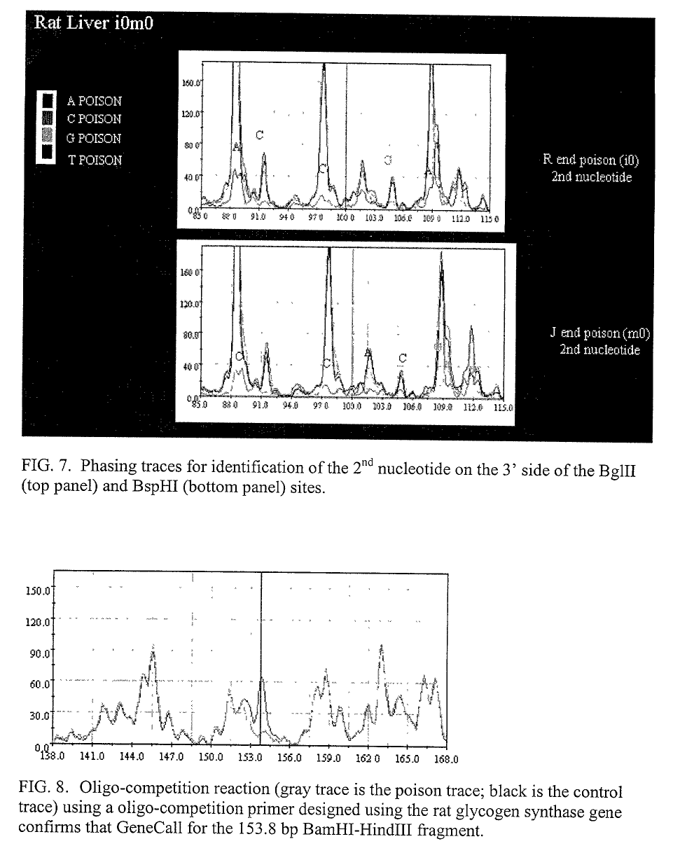

For nucleotide oligo-competition at the second position, primers M0J2A, M0J2C, M0J2G, and M0J2T were used in oligo-competition reactions for the BspHI restriction enzyme site on the J side, and primers I0R2A, I0R2C, I0R2G, and I0R2T, for oligo-competition reactions for the BglII restriction site on the R side. FIG. 7 shows, for example, that for the QEA peak at 88.2 bp, the second nucleotide on the J side is a cytosine (C), and on the R side is an adenine (A). Corresponding results are provided for the other QEA peaks in FIG. 7 as well.

Oligo-competition Utility

Genecalling lists are refined by a predicted factor of 256 with the additional information provided by oligo-competing for two cycles at both the J and R subsequences. As an example of a practical application of this specificity, a HindIII-BamHI double restriction digest of rat liver cDNA provided a 153.8 bp fragment for which the original GeneCalling list has 10 candidate genes whose subsequence-length combinations match the experimental information. Of the 10 candidate genes, only one, Rat Glycogen Synthase, matches the oligo-competition data provided by two cycles of phasing for each of the J and R subsequences for this fragment. Thus an unambiguous matching of gene to a fragment is provided as a result of the oligo-competing process. This matching is confirmed by a oligo-competition experiment using an oligonucleotide incorporating bases identified by the extended oligo-competition process described for FIGS. 6 and 7 and in this paragraph (see FIG. 8). The upper trace at 153.8 bp in FIG. 8 is the control trace in the absence of the oligo-competition oligonucleotide, and the lower trace is obtained in the presence of and excess of the oligonucleotide.

### Example 3

### Identification of Poisoned Bands

Oligo-competition (automated or manual or both) is a process of finding a limited nucleotide sequence of the cDNA fragments adjacent to their known cut sites. The sequence of interest is determined by altering the amounts of cDNA fragments in the oligo-competition PCR process using one of the four (for a single position) sequence-specific oligo-competition primers (see Detailed Description and Example 2) and by analyzing the resulting electrophoresis traces of the oligo-competition PCR products in terms of their intensities as functions of the electrophoresis mobilities expressed in terms of cDNA fragment lengths (bp). The oligo-competition nucleotides (oligo-competition sequence) are identified based on the differences in the oligo-competition PCR amplification, which in turn are determined by the differences in the intensities of the traces in the narrow neighborhood of the peak corresponding to a given cDNA fragment of the PCR product. The oligo-competition PCR process may be designed so that the intensity of the poisoned cDNA fragments in the oligo-competition PCR product will be reduced (negative oligo-competition) or increased (positive oligo-competition) with regards to the intensity corresponding to the non-poisoned fragment of the same length and cut sequence. In the following the negative oligo-competition algorithm is described, while the differences pertaining to the positive oligo-competition algorithm are noted in parentheses where applicable. The analysis of the oligo-competition data can be applied to the oligo-competition electrophoresis traces alone (up to four traces corresponding to four possible nucleotides A, C, G, and T in a given nucleotide position), or to the oligo-competition electrophoresis traces combined with the electrophoresis traces corresponding to the initial mixture of the cDNA fragments used as input into oligo-competition PCR process (up to five traces). This analysis may consist of the following steps.

1) Normalization, Averaging and Scaling of the Intensities of the Electrophoresis Traces.

The intensity of the electrophoresis trace in principle characterizes the amount of the cDNA fragments in PCR product as a function of their length. However, the value of the trace intensity is influenced by several undesired factors acting at different stages of the oligo-competition process. These factors include but are not limited to (i) uncertainty in the initial amount of the cDNA fragments used in oligo-competition PCR, (ii) variations in oligo-competition PCR amplification, which depends on oligo-competition PCR primers, fragment length and other parameters of the PCR process, (iii) electrophoresis instrument noise etc. The influence of these factors on the oligo-competition traces is reduced by normalization and scaling (see WO00/41122). Normalization and scaling can be applied to oligo-competition traces alone or to the oligo-competition traces combined with the traces of initial cDNA fragments.

2) Peak Finding.

The traces refined on step 1 are analyzed to determine the peaks (local intensity maxima) that identify cDNA by both their cut sequences and length. For each pair of cut sites, possible options include but are not limited to (i) analysis of the individual oligo-competition traces, each corresponding to a specific oligo-competition PCR primer, (ii) analysis of the composite trace representing the average of all oligo-competition traces, (iii) analysis of the composite trace representing the average of all oligo-competition traces and trace of the initial cDNA fragments, (iv) analysis of the traces of initial cDNA fragments. The peak finding algorithm scans a given trace for maxima, identified by a predetermined set of conditions, such as the shape of the trace in a given number of consecutive points, the signal/noise ratio and others.

3) Difference Finding.

For each of the peaks found on step 2, the normalized and scaled oligo-competition traces are ranked in ascending (descending, for positive oligo-competition) order of their maximum intensities determined within a narrow neighborhood of the position of a given peak. The peak is then considered to be poisoned if certain conditions with regards to the interrelationships of the ranked intensities are met. Possible options for these conditions in negative PhaseCalling include but are not limited to:

(i) the intensity of n (n<4) first ranked oligo-competition traces are at least k-times (k>1) lower (higher, for positive PC) than that of any other oligo-competition trace within the location of the given peak. The oligo-competition primers corresponding to each of these oligo-competition traces determine 1 to n poisoned nucleotides and their position relative to the cDNA fragment's cut site.

(ii) the intensity of n (n 4) first ranked oligo-competition traces are at least k-times (k>1) lower (higher, for positive PC) than that of the trace of the non-PC-treated cDNA fragments within the location of the given peak. In this case, the oligo-competition primers corresponding to each of these oligo-competition traces determine 1 to n poisoned nucleotides and their position relative to the cDNA fragment's cut site.

The values of n and k can be determined empirically and optimized to better reproduce the sequences of the cDNA fragments. In pilot oligo-competition software the values of n and k were fixed at 2 and 1.5 correspondingly.

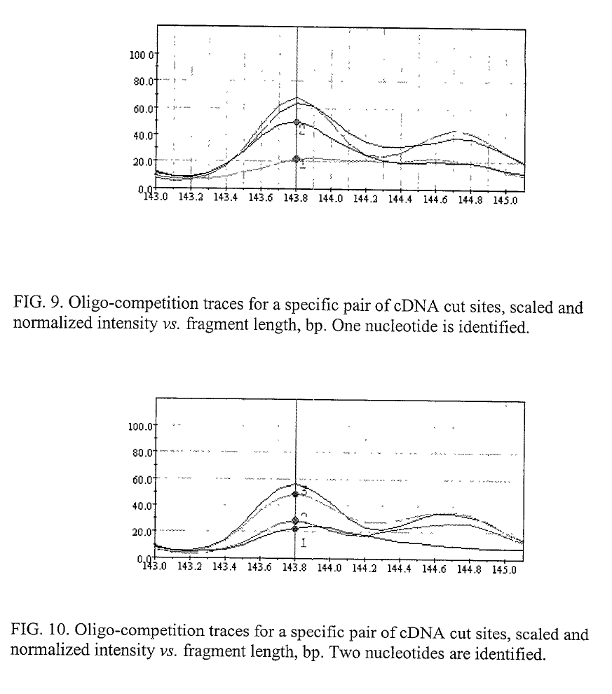

FIGS. 9 and 10 illustrate examples of the oligo-competition algorithm applied to four oligo-competition traces of the negative oligo-competition PCR products for cDNA fragments 143.8 bp long. The red vertical line identifies the peak of interest, and the numbers identify the ranking order of the oligo-competition traces.

In FIG. 9 the intensity of the green oligo-competition trace is 1.5 times lower than that of the first trace above it, so that it was determined that this cDNA fragment has a nucleotide G immediately adjacent to the cut sequence (green trace corresponds the oligo-competition primer specific to the G nucleotide in the first position with regards to the cut site).

In FIG. 10 the intensity of both black and red oligo-competition traces are at least 1.5 times lower than that of any trace above them, so that it was determined that the cDNA fragments, characterized by this particular cut sequence and length, have T and C nucleotides that are located next to the cut sequence (black and red traces correspond the oligo-competition primer specific to the T and C nucleotides adjacent to the cut site respectively).

### Example 4

### Application of Trace-oligo-competition Data To Improve Confirmation Efficiency

A total of ten different trace oligo-competition projects were done in three different organisms as shown in Table 4. Additional sequence information of up to four nucleotides adjacent to the restriction enzyme recognition sites of the bands was generated for the bands. This information was used to optimize gene calls for the identified fragments. The gene calls were then compared with results from further confirmations resulting from procedures such as oligo-competition (see Background of the Invention) or sequencing. The confirmation requests were categorized into five groups based on the number of nucleotides added in the trace oligo-competition matches between the bands and their gene calls (termed “Trace Oligo-competition_Score”). The score ranges from 0 to 4 nucleotides added. The effectiveness of this process was assessed by evaluating the percentage of the positive confirmations of referred to the total number of confirmation requests in each category. These ratios were also compared to earlier historical data where confirmations were done without trace oligo-competition.

TABLE 4
List of ten trace oligo-competition projects completed in
three different organisms (as indicated by Organism ID) and
various tissues (as indicated by Tissue ID)

| Trace oligo- | | |
|---|---|---|
| competition_Project | ORGANISM ID | TISSUE ID |
| 1 | 9 | 19 |
| 2 | 9 | 43 |
| 3 | 9 | 45 |
| 4 | 9 | 47 |
| 5 | 9 | 62 |
| 6 | 9 | 65 |
| 7 | 9 | 74 |
| 8 | 9 | 144 |
| 9 | 17 | 54 |
| 10 | 26 | 19 |

Results:

1. Trace Oligo-competition Data Improves Confirmation Efficiency

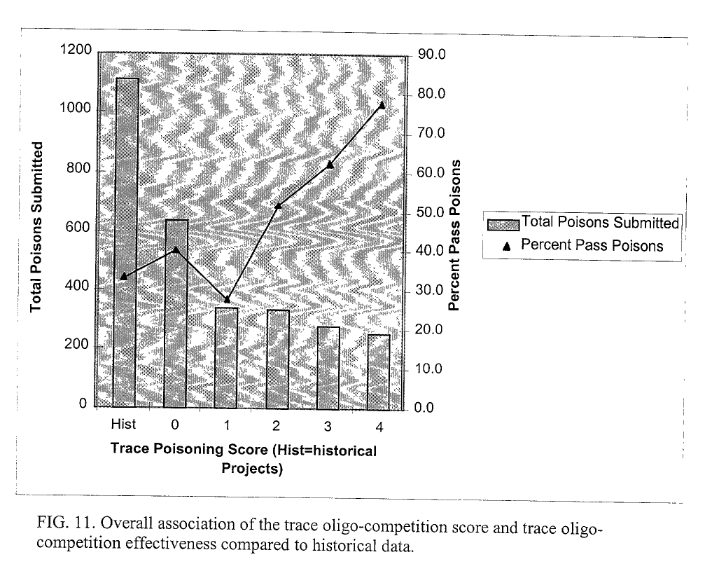

The impact of the Trace Oligo-competition Scores on confirmation efficiency in ten different trace oligo-competition projects is summarized in FIG. 11. 1828 confirmations were submitted from the ten trace oligo-competition projects. These confirmations were categorized based on the number of trace oligo-competition-nucleotide matches between the bands and their gene calls (Trace Oligo-competition Scores). The trace oligo-competition efficiency was measured as the percentage of the trace oligo-competition runs that were confirmed by oligo-competition to the total number of confirmation requests in each category. The results were also compared to the confirmation efficiency of another 1108 historical samples where no trace oligo-competition data was generated.

It is seen from FIG. 11 that the overall trace oligo-competition effectiveness increased with the number of nucleotides employed in the trace oligo-competition procedure. With a Trace Oligo-competition Score of 1, the confirmation efficiency was lower than that when no matches were found or when compared to the confirmation efficiency of the historical data. This may be due to an experimenter's bias towards the selection of specific band-to-gene associations when trace oligo-competition data was not available (Historical or Score=0). On average the trace oligo-competition efficiency increased by 9.3% per base over the full range from 0 to 4 (40.2% to 77.5%). This general trend was consistent across all the ten trace oligo-competition projects (see Table 5, showing a detailed breakdown of trace oligo-competition effectiveness in various projects). The variation in confirmation efficiency between trace oligo-competition projects for a given Trace Oligo-competition Score can be explained, at least in part, by the quality, tissue specificity and redundancy of the sequence databases.

TABLE 5
Association of the Trace Oligo-competition Score and the oligo-competition success in
several trace oligo-competition projects.

| TRACE | | | Trace | | | | |
|---|---|---|---|---|---|---|---|
| OLIGO- | | | Oligo- | | | | |
| COMPETITION | ORGANISM | TISSUE | competition | | | | Pass |
| Tissue Ck | ID | ID | Score | NO PASS | PASS | TOTAL | % |
| 0 | Several | Several | Hist | 740 | 368 | 1108 | 33.2 |
| 1 | 9 | 19 | 0 | 30 | 10 | 40 | 25.0 |
| 1 | 9 | 19 | 1 | 32 | 15 | 47 | 31.9 |
| 1 | 9 | 19 | 2 | 24 | 12 | 36 | 33.3 |
| 1 | 9 | 19 | 3 | 20 | 28 | 48 | 58.3 |
| 1 | 9 | 19 | 4 | 4 | 23 | 27 | 85.2 |
| 1 | 9 | 43 | 0 | 74 | 15 | 89 | 16.9 |
| 1 | 9 | 43 | 1 | 99 | 24 | 123 | 19.5 |
| 1 | 9 | 43 | 2 | 33 | 18 | 51 | 35.3 |
| 1 | 9 | 43 | 3 | 16 | 21 | 37 | 56.8 |
| 1 | 9 | 43 | 4 | 18 | 29 | 47 | 61.7 |
| 1 | 9 | 45 | 0 | 20 | 27 | 47 | 57.4 |
| 1 | 9 | 45 | 1 | 7 | 6 | 13 | 46.2 |
| 1 | 9 | 45 | 2 | 5 | 5 | 10 | 50.0 |
| 1 | 9 | 45 | 3 | 1 | 4 | 5 | 80.0 |
| 1 | 9 | 45 | 4 | 1 | 4 | 5 | 80.0 |
| 1 | 9 | 47 | 0 | 19 | 17 | 36 | 47.2 |
| 1 | 9 | 47 | 1 | 8 | 6 | 14 | 42.9 |
| 1 | 9 | 47 | 2 | 7 | 7 | 14 | 50.0 |
| 1 | 9 | 47 | 3 | 13 | 18 | 31 | 58.1 |
| 1 | 9 | 47 | 4 | 9 | 20 | 29 | 69.0 |
| 1 | 9 | 62 | 0 | 46 | 15 | 61 | 24.6 |
| 1 | 9 | 62 | 1 | 23 | 8 | 31 | 25.8 |
| 1 | 9 | 62 | 2 | 20 | 20 | 40 | 50.0 |
| 1 | 9 | 62 | 3 | 9 | 35 | 44 | 79.5 |
| 1 | 9 | 62 | 4 | 13 | 55 | 68 | 80.9 |
| 1 | 9 | 65 | 0 | 1 | | 1 | 0.0 |
| 1 | 9 | 65 | 1 | 10 | 4 | 14 | 28.6 |
| 1 | 9 | 65 | 2 | 9 | 11 | 20 | 55.0 |
| 1 | 9 | 65 | 3 | 8 | 11 | 19 | 57.9 |
| 1 | 9 | 65 | 4 | 1 | 16 | 17 | 94.1 |
| 1 | 9 | 74 | 0 | 22 | 22 | 44 | 50.0 |
| 1 | 9 | 74 | 1 | 16 | 4 | 20 | 20.0 |
| 1 | 9 | 74 | 2 | 28 | 17 | 45 | 37.8 |
| 1 | 9 | 74 | 3 | 17 | 18 | 35 | 51.4 |
| 1 | 9 | 74 | 4 | 5 | 15 | 20 | 75.0 |
| 1 | 9 | 144 | 0 | 115 | 124 | 239 | 51.9 |
| 1 | 9 | 144 | 1 | 28 | 11 | 39 | 28.2 |
| 1 | 9 | 144 | 2 | 9 | 30 | 39 | 76.9 |
| 1 | 9 | 144 | 3 | 9 | 17 | 26 | 65.4 |
| 1 | 9 | 144 | 4 | | 15 | 15 | 100.0 |
| 1 | 9 | 146 | 0 | 19 | 6 | 25 | 24.0 |
| 1 | 9 | 146 | 1 | 2 | | 2 | 0.0 |
| 1 | 9 | 146 | 2 | 2 | 1 | 3 | 33.3 |
| 1 | 9 | 146 | 3 | 2 | 1 | 3 | 33.3 |
| 1 | 9 | 146 | 4 | | 1 | 1 | 100.0 |
| 1 | 17 | 54 | 0 | 22 | 7 | 29 | 24.1 |
| 1 | 17 | 54 | 1 | 13 | | 13 | 0.0 |
| 1 | 17 | 54 | 2 | 15 | 6 | 21 | 28.6 |
| 1 | 17 | 54 | 3 | 8 | 7 | 15 | 46.7 |
| 1 | 17 | 54 | 4 | 6 | 13 | 19 | 68.4 |
| 1 | 26 | 19 | 0 | 11 | 12 | 23 | 52.2 |
| 1 | 26 | 19 | 1 | 6 | 15 | 21 | 71.4 |
| 1 | 26 | 19 | 2 | 7 | 43 | 50 | 86.0 |
| 1 | 26 | 19 | 3 | 1 | 11 | 12 | 91.7 |
| 1 | 26 | 19 | 4 | | 5 | 5 | 100.0 |

| | | | | |
|---|---|---|---|---|
| All | 1683 | 1253 | 2936 | 42.7 |

Trace Oligo-competition Tissue Ck = Whether trace oligo-competition data set is available, 0 = No, 1 = Yes.
Trace Oligo-competition Score = Number of nucleotide matches between trace oligo-competition data of the band and the sequence of the gene call.
NOPASS = Failure to confirm the Band to Gene association by ′Oligo-competition′ (Competitive PCR).
PASS = Positive confirmation of the Band to Gene association by ′Oligo-competition′ (Competitive PCR).
TOTAL = Total number of oligo-competitions submitted.
Pass % = Percentage of the PASS to TOTAL.
Hist = Historical confirmation data without any trace oligo-competition projects.

II. Trace Oligo-competition Complements Other Technologies in Further Improving Confirmation Efficiency

A total of 1073 confirmations that were done in various projects using the gene calls from a CuraGen Corporation proprietary sequence database were used to evaluate the effectiveness of the trace oligo-competition data. Among the 1073 confirmations, trace oligo-competition data were available for 688 confirmation requests. The remaining 385 confirmation requests were treated as historical data where confirmations were done only with the proprietary database. The trace oligo-competition data from different trace oligo-competition projects was used to identify the Trace Oligo-competition Score for each confirmation done. As described before, the confirmation efficiency was measured as the percentage of the positive confirmations to the total number of confirmation requests in each category. The results were also compared to the confirmation efficiency of the 385 historical confirmations where no trace oligo-competition data could be used.

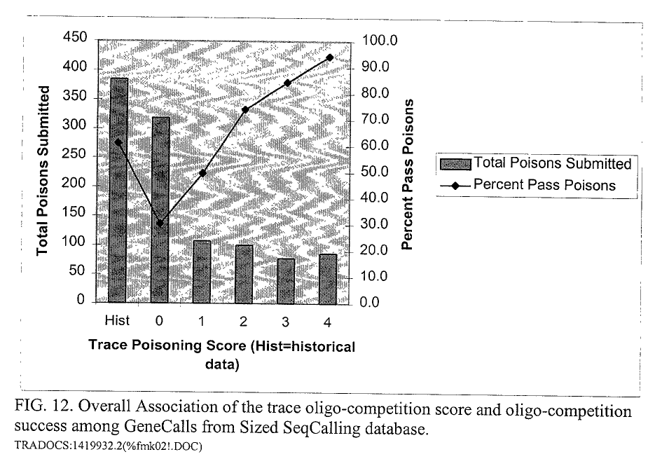

The overall effectiveness of trace oligo-competition on further improving confirmation efficiency among gene calls from the proprietary database is shown in FIG. 12. The overall confirmation efficiency using Sized SeqCalling™ database in the historical data was 61% when the proprietary database was used within the same tissue and developmental stage. The confirmation efficiency decreases from 61% in the historical data to 30% among confirmations requested having Scores=0 in the trace oligo-competition projects. This reduction is due to the tissue and developmental stage differences between the samples where the proprietary database was generated and those where the gene calls were used for confirmation. The trace oligo-competition effectiveness increases with Trace Oligo-competition Score. With a match of 2 or more nucleotides, the confirmation efficiency was more than the confirmation efficiency observed in the historical data. These results demonstrate that trace oligo-competition complements the use of the proprietary database and further improves the confirmation efficiency. The results were consistent in all the trace oligo-competition Projects where confirmations were submitted using the proprietary database (see Table 6, showing a detailed breakdown of trace oligo-competition effectiveness in various projects).

TABLE 6
Association of the Trace Oligo-competition Score and the oligo-competition success
among GeneCalls from Sized SeqCalling database in several trace Oligo competition Projects.

| TRACE | | | Trace | | | | |
|---|---|---|---|---|---|---|---|
| OLIGO- | | | Oligo- | | | | |
| COMPETITION | ORGANISM | TISSUE | competition | | | | |
| Tissue₋Ck | ID | ID | n Score | NOPASS | PASS | TOTAL | Pass₋perc |
| 0 | Several | Several | Hist | 150 | 235 | 385 | 61.0 |
| 1 | 9 | 19 | 0 | 25 | 23 | 48 | 47.9 |
| 1 | 9 | 19 | 1 | 7 | 11 | 18 | 61.1 |
| 1 | 9 | 19 | 2 | 1 | 18 | 19 | 94.7 |
| 1 | 9 | 19 | 3 | | 17 | 17 | 100.0 |
| 1 | 9 | 19 | 4 | | 22 | 22 | 100.0 |
| 1 | 9 | 43 | 2 | | 2 | 2 | 100.0 |
| 1 | 9 | 43 | 3 | | 1 | 1 | 100.0 |
| 1 | 9 | 43 | 4 | | 1 | 1 | 100.0 |
| 1 | 9 | 45 | 0 | 17 | 3 | 20 | 15.0 |
| 1 | 9 | 45 | 1 | 5 | 6 | 11 | 54.5 |
| 1 | 9 | 45 | 2 | 2 | 4 | 6 | 66.7 |
| 1 | 9 | 45 | 3 | | 4 | 4 | 100.0 |
| 1 | 9 | 45 | 4 | | 2 | 2 | 100.0 |
| 1 | 9 | 47 | 0 | 37 | 13 | 50 | 26.0 |
| 1 | 9 | 47 | 1 | 10 | 8 | 18 | 44.4 |
| 1 | 9 | 47 | 2 | 5 | 7 | 12 | 58.3 |
| 1 | 9 | 47 | 3 | 2 | 9 | 11 | 81.8 |
| 1 | 9 | 47 | 4 | | 19 | 19 | 100.0 |
| 1 | 9 | 62 | 2 | 1 | | 1 | 0.0 |
| 1 | 9 | 62 | 3 | 1 | 2 | 3 | 66.7 |
| 1 | 9 | 62 | 4 | 1 | 2 | 3 | 66.7 |
| 1 | 9 | 65 | 0 | 6 | 14 | 20 | 70.0 |
| 1 | 9 | 74 | 0 | 3 | 1 | 4 | 25.0 |
| 1 | 9 | 74 | 1 | 1 | 1 | 2 | 50.0 |
| 1 | 9 | 74 | 2 | 1 | 9 | 10 | 90.0 |
| 1 | 9 | 74 | 3 | 6 | 5 | 11 | 45.5 |
| 1 | 9 | 74 | 4 | | 4 | 4 | 100.0 |
| 1 | 9 | 144 | 0 | 134 | 42 | 176 | 23.9 |
| 1 | 9 | 144 | 1 | 31 | 27 | 58 | 46.6 |
| 1 | 9 | 144 | 2 | 16 | 34 | 50 | 68.0 |
| 1 | 9 | 144 | 3 | 3 | 27 | 30 | 90.0 |
| 1 | 9 | 144 | 4 | 4 | 31 | 35 | 88.6 |

| | | | | |
|---|---|---|---|---|
| All | 469 | 604 | 1073 | 56.3 |

Trace Oligo-competition Tissue Ck = Whether trace oligo-competition data set is available, 0 = No, l = Yes.
Trace Oligo-competition Score = Number of nucleotide matches between trace oligo-competition data of the band and the sequence of the gene call.
NOPASS = Negative confirmation of the Band to Gene association by ′Oligo-competition′ (Competitive PCR).
PASS = Positive confirmation of the Band to Gene association by ′Oligo-competition′ (Competitive PCR).
TOTAL = Total number of oligo-competitions submitted.
Pass % = Percentage of the PASS to TOTAL.
Hist = Historical confirmation data without any trace oligo-competition projects.

### Equivalents

From the foregoing detailed description of the specific embodiments of the invention, it should be apparent that particular novel compositions and methods involving nucleic acids, polypeptides, antibodies, detection and treatment have been described. Although these particular embodiments have been disclosed herein in detail, this has been done by way of example for purposes of illustration only, and is not intended to be limiting with respect to the scope of the appended claims that follow. In particular, it is contemplated by the inventors that various substitutions, alterations, and modifications may be made as a matter of routine for a person of ordinary skill in the art to the invention without departing from the spirit and scope of the invention as defined by the claims. Indeed, various modifications of the invention in addition to those described herein will become apparent to those skilled in the art from the foregoing description and accompanying figures. Such modifications are intended to fall within the scope of the appended claims.

What is claimed is:

1. A method for identifying, classifying or quantifying one or more nucleic acids in a sample comprising a plurality of nucleic acids having different nucleotide sequences, the method comprising:
(a) probing said sample with one or more recognition means wherein each recognition means recognizes a different target nucleotide subsequence or a different set of target nucleotide subsequences to provide one or more targeted nucleic acids;
(b) generating one or more first signals from said sample probed by said recognition means, each generated first signal arising from a targeted nucleic acid in said sample and comprising a representation of (i) the length between occurrences of target subsequences in said targeted nucleic acid, and (ii) the identities of said target subsequences in said targeted nucleic acid or identities of said target subsequences among which are included the target subsequences in said targeted nucleic acid;
(c) selecting one or more targeted nucleic acids based on their corresponding first signals;
(d) extending sequence information from one or more target subsequences in said selected targeted nucleic acid by one or more nucleotides providing one or more extended subsequences under conditions that generate one or more second signals arising from said selected targeted nucleic acid, at least one of whose subsequences has been extended, in said sample, wherein said second signal comprises a representation of (i) the length between occurrences of target subsequences, at least one of which has been extended, in said nucleic acid, and (ii) the identities of said selected target subsequences, at least one of which has been extended, in said selected targeted nucleic acid or identities of said target subsequences, at least one of which has been extended, among which are included the target subsequences in said selected targeted nucleic acid; and
(e) searching a nucleotide sequence database to determine sequences that match or the absence of any sequences that match one or more or of said selected targeted nucleic acids having at least one extended subsequence and represented by said generated second signals, said database comprising a plurality of known nucleotide sequences of nucleic acids that may be present in the sample, wherein a sequence from said database is determined to match said selected targeted nucleic acid providing a generated second signal when the sequence from said database has both (i) the same length between occurrences of target subsequences, at least one of which has been extended, as is represented by the generated signal, and (ii) the same target subsequences, at least one of which has been extended, as are represented by the generated signal, or target subsequences, at least one of which has been extended, that are members of the same sets of target subsequences represented by the generated signal,
whereby a matched nucleic acid in said sample is identified, classified, or quantified.

2. The method of claim 1 wherein said second generated signal is a negative oligo-competition signal.

3. The method of claim 1 wherein said second generated signal is a positive oligo-competition signal.

4. The method of claim 2 wherein the extending of the sequence information comprises contacting the nucleic acid sample with a mixture of oligonucleotides comprising (i) a set of labeled primers each of whose nucleotide sequences comprises a target subsequence and (ii) an unlabeled primer whose sequence comprises one of the target subsequences identified in (i) followed by at least one additional nucleotide.

5. The method of claim 3 wherein the extending of the sequence information comprises contacting the nucleic acid sample with a mixture of oligonucleotides comprising (i) a set comprising a first unlabeled primer and a second unlabeled primer each of whose nucleotide sequence comprises a target subsequence and (ii) a set comprising a labeled third primer whose sequence comprises the subsequence of the first unlabeled primer and a labeled fourth primer whose sequence comprises the subsequence of the second unlabeled primer extended by at least one nucleotide.

6. The method of claim 1 wherein at least one of said generated signals corresponds to a sequence having a size and target subsequence of a sequence present in said sequence database.

7. The method of claim 1 wherein said method additionally includes
recovering a fragment of a nucleic acid in the sample which generates said signal;
sequencing said fragment to determine at least a partial sequence for said fragment; and
verifying that said sample comprises a nucleic acid having a sequence comprising at least a portion of said determined sequence.

8. The method of claim 1 wherein said plurality of nucleic acids are DNA.

9. The method of claim 8, wherein said probing comprises:
digesting the sample with one or more restriction endonucleases, said restriction endonucleases having recognition sites that are said target subsequences and leaving single-stranded nucleotide overhangs on the digested ends;
hybridizing double-stranded adapter nucleic acids with the digested sample fragments, said adapter nucleic acids having an end complementary to one of said single-stranded overhangs; and
ligating the complementary ends end the of adapter nucleic acids to the complementary 5′-end of a strand of the digested sample fragments to form ligated nucleic acid fragments.

10. The method of claim 7, wherein said plurality of nucleic acids are RNA.

11. A method for extending the sequence in a length-subsequence combination of one or more nucleic acids in a sample comprising a plurality of nucleic acids having different nucleotide sequences, said method comprising:
(a) probing said sample with one or more recognition means wherein each recognition means recognizes a different target nucleotide subsequence or a different set of target nucleotide subsequences to provide one or more targeted nucleic acids;
(b) generating one or more first signals from said sample probed by said recognition means, each generated first signal arising from a targeted nucleic acid in said sample and comprising a representation of (i) the length between occurrences of target subsequences in said targeted nucleic acid, and (ii) the identities of said target subsequences in said targeted nucleic acid or identities of said target subsequences among which are included the target subsequences in said targeted nucleic acid;
(c) selecting one or more targeted nucleic acids based on their corresponding first signals; and
(d) extending sequence information from one or more target subsequences in said targeted nucleic acid by one or more nucleotides providing one or more extended subsequences under conditions that generate one or more second signals arising from selected targeted nucleic acid in said sample at least one of whose subsequences has been extended, wherein said second signal comprises a representation of (i) the length between occurrences of target subsequences, at least one of which has been extended, in said nucleic acid, and (ii) the identities of said target subsequences, at least one of which has been extended, in said selected targeted nucleic acid or identities of said target subsequences, at least one of which has been extended, among which are included the target subsequences in said selected targeted nucleic acid;
whereby a matched nucleic acid in said sample has an extended sequence in said length-subsequence combination.

12. The method of claim 11 wherein said second generated signal is a negative oligo-competition signal.

13. The method of claim 11 wherein said second generated signal is a positive oligo-competition signal.

14. The method of claim 12 wherein the extending of the sequence information comprises contacting the nucleic acid sample with a mixture of oligonucleotides comprising (i) a set of labeled primers each of whose nucleotide sequences comprises a target subsequence and (ii) an unlabelled primer whose sequence comprises one of the target subsequences identified in (i) followed by at least one additional nucleotide.

15. The method of claim 13 wherein the extending of the sequence information comprises contacting the nucleic acid sample with a mixture of oligonucleotides comprising (i) a set comprising a first unlabeled primer and a second unlabeled primer each of whose nucleotide sequence comprises a target subsequence and (ii) a set comprising a labeled third primer whose sequence comprises the subsequence of the first unlabeled primer and a labeled fourth primer whose sequence comprises the subsequence of the second unlabeled primer extended by at least one nucleotide.
